(19) United States
(12) Patent Application Publication
(10) Pub. No.: US 2022/0184621 A1
(43) Pub. Date: Jun. 16, 2022

(54) INTEGRATED MICROFLUIDIC CHIP FOR CELL IMAGING AND BIOCHEMICAL DETECTION AND METHOD USING THE SAME

(71) Applicant: NATIONAL CENTRAL UNIVERSITY, Taoyuan City (TW)

(72) Inventors: CHEN HAN HUANG, Taoyuan City (TW); CHUN SAN TAI, Taoyuan City (TW); TING YI LIN, Taoyuan City (TW)

(73) Assignee: NATIONAL CENTRAL UNIVERSITY, Taoyuan City (TW)

(21) Appl. No.: 17/543,728

(22) Filed: Dec. 6, 2021

Related U.S. Application Data
(60) Provisional application No. 63/124,958, filed on Dec. 14, 2020.

(51) Int. Cl. B01L 3/00; G01N 27/02

(52) U.S. Cl. CPC B01L 3/502761; B01L 3/502715; G01N 27/02; B01L 2200/0684; B01L 2300/0645; B01L 2300/0636; B01L 2200/16; B01L 2300/0887

(57) ABSTRACT

An integrated chip having an integrated function capable of providing cell image and biochemical detection is provided and includes a sequentially stacked and sealed laminate set. The laminate set includes an upper laminate, a middle laminate and a lower laminate. The upper laminate composed of one or more plates and having one or more holes for sample injection and sample or air discharging of. The middle laminate composed of one or more plates includes at least two hollow structures that define an imaging chamber and a biochemical detection area. The lower laminate includes at least one filtering element and at least one electrode sensing element disposed in the biochemical detection. The filtering element is for blocking suspended particles, and the electrode sensing element has electrode terminals for connecting to instruments or equipment to perform the measurements and analysis of electrochemistry and impedance.

### BACKGROUND OF THE INVENTION

### 1. Field of the Invention

The present invention relates to an integrated chip that has an integrated function capable of providing cell image and biochemical detection, and more particularly relates to a microfluidic biological detection chip that includes a cell fluid chamber and an electrochemistry detection area.

### 2. Description of Related Art

Biochemical characteristic analysis and the image for a specific cell is generally applied in biomedical detection and can provide qualitative and quantitative research for cell and molecule target and medical efficacy evaluation related, etc.

A microfluidic chip can be used for detecting microparticles, such as cells, genetic materials, and proteins, etc., and has been utilized in the fields such as chemical analysis, biomedicine, and environmental monitoring, etc. In the testing process, a sample with microparticles in microfluidic channels needs to be steadily positioned on the center to increase the accuracy of the detection. In general, the positioning manner includes hydrodynamic positioning and acoustic positioning, etc.

However, the conventional chips for biological detection rarely have an integrated function capable of providing image analysis and biological detection simultaneously. Therefore, the present invention provides an integrated chip with cellular molecule imaging and biological detection by utilizing the design of functional microfluidic channels. The chip includes a cell liquid chamber for air releasing and electrodes for electrochemistry detection section. The chamber can provide the storage of a sampled fluid biopsy and the usage of image capturing. The electrode portions in the electrochemistry detection section can sense the bio-electrochemical signals, so the multi-functional microfluidic channels of the chip can be utilized to provide image detection and biochemistry related detection simultaneously.

### SUMMARY OF THE INVENTION

In one aspect, the present invention relates to an integrated chip for providing cell image and biochemical detection including a sequentially stacked and sealed laminate set. The laminate set includes an upper laminate, a middle laminate and a lower laminate. The first laminate composed of one or more plates haves one or more holes penetrating the upper laminate, the holes used for sample injection, and sample or air discharging, respectively. The middle laminate composed of one or more plates includes at least two hollow structures that define an imaging chamber and a biochemical detection area, respectively. The lower laminate includes at least one set of a filtering element and electrode sensing element that is disposed in the biochemical detection. The filtering element is for blocking suspended particles, and the electrode sensing element has an electrode section and electrode terminals that can provide connection to instruments or equipment to perform the measurements and analysis of electrochemistry and impedance.

The integrated chip of the present invention can detect the morphology of a cell and the microparticles contained therein (a specific genetic material, protein, etc.) simultaneously. The detection manners generally include the method such as electrical impedance detection, fluorescence detection, light scattering, microscopic imaging, etc.

In the preferred embodiment of the present invention, the plates of the laminate set can be made of the materials selected from light penetrable (preferably transparent) glass, plastic, acrylic, etc., and the thickness of each plate is between 50-300 micrometers.

In the preferred embodiment of the present invention, the imaging chamber can be connected to an image monitoring device that includes a microscopic image receiver and an image analysis device.

In the preferred embodiment of the present invention, the electrode sensing element includes at least a pair of microelectrodes on which electrical signals with different amplitudes and frequencies are applied (e.q., cyclic voltammetry (CV) method, the chronoamperometry method, etc.) to perform the measurements of electrochemistry.

In the preferred embodiment of the present invention, a molecule for capturing, e.q., biomolecules such as an antibody, antigen, nucleic acid, protein, etc., is modified in the electrode section to allow a target molecule present in a sample to be bound with the molecule for capturing. The electrochemical measurements for the target molecule captured on the chip can be performed using the electrochemical characteristics of the target molecule. Alternatively, a corresponding molecule carrying a detectable material, e.q., biomolecules such as an antibody, antigen, nucleic acid, protein, etc. is further introduced and bound again to perform the electrochemical measurements and analysis of the corresponding molecule via the electrode terminals.

### BRIEF DESCRIPTION OF THE DRAWINGS

The invention as well as a preferred mode of use, further objectives and advantages thereof will be best understood by reference to the following detailed description of illustrative embodiments when read in conjunction with the accompanying drawings, wherein:

FIG. 1 is a structural illustration of an integrated microfluidic chip for cell imaging and biochemical detection according to embodiment 1 of the present invention.

FIG. 2 is a top view photo of an integrated microfluidic chip 100 for cell imaging and biochemical detection. The chip has empty slots for capturing cell or particle image and for performing electrochemical detection, i.e., an imaging chamber 1051 and a biochemical detection area 1061, respectively.

FIG. 3 is an exemplary figure of filtering elements of the integrated microfluidic chip for cell imaging and biochemical detection according to the present invention. The filtering elements can include a filtering area composed of one or more microarrays in the form of a pillar (a), fence (b), or sieve (c).

FIG. 4 is an illustration of modifications and detection being performed on the electrode sensing element of the integrated microfluidic chip for cell imaging and biochemical detection according to the present invention. FIG. 4(a) is an illustration of a molecule for capturing (e.q., an antibody) being modified first on the electrode sensing element; FIG. 4(b) is an illustration of a target molecule (e.q., an antigen) being bound with the molecule for capturing; 4(c) is an illustration of a molecular (e.q., a secondary antibody) carrying a detectable material being bound with the captured target molecule; and FIG. 4(d) is an illustration of electrochemical measurements and analysis being performed, on an electrode section of the sensing element, for the detectable material using its electrochemical characteristics.

FIG. 5 are the imaging results of blood cells (A) and nerve cells (B). Samples are injected into the chips of the present invention in which the cells are dispersed in an imaging chamber of the chips, and images of the chips are then captured in a microscope system.

FIG. 6 is a result of performing electrochemical measurement for the chip of the present invention. The solution of potassium hexacyanoferrate(III) is introduced into the chip, and the measurement is performed utilizing the cyclic voltammetry (CV) method in electrochemistry via the electrode terminals of the chip that are connected to an electrochemical sensing instrument.

FIG. 7 is a result of performing electrochemical measurement for the chip of the present invention. A different concentration of protein kinase (AKT1) is introduced into the chip, and the measurement is performed utilizing the chronoamperometry method in electrochemistry via the electrode terminals of the chip that are connected to an electrochemical sensing instrument.

### DETAILED DESCRIPTION OF THE PREFERRED EMBODIMENT

These and other aspects of the embodiments herein will be better appreciated and understood when considered in conjunction with the following description and the accompanying drawings.

The embodiments below are used to illustrate the present invention and are not considered as a limitation to the scope of the present invention. Unless specifically designated, the techniques used in the embodiments are ordinary skills known to the skilled in the art, and all materials are commercially available.

Embodiment 1: integrated microfluidic chip for cell imaging and biochemical detection.

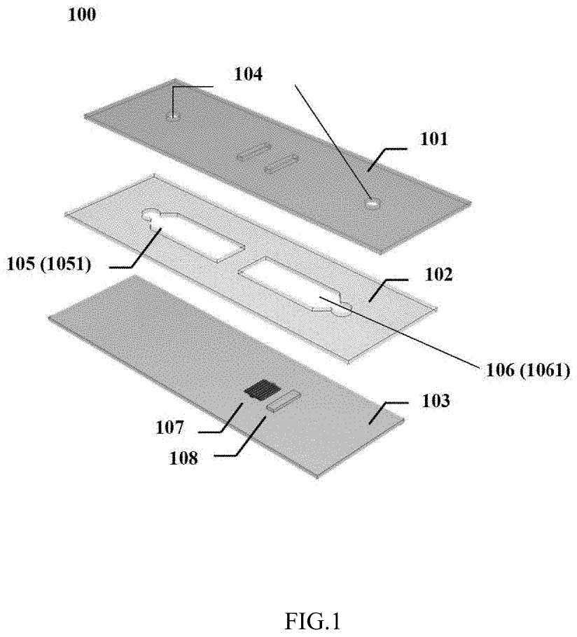

Referring to FIG. 1, an integrated microfluidic chip 100 for cell imaging and biochemical detection provided by this embodiment is composed of several laminates: an upper laminate 101, a middle laminate 102 and a lower laminate 103. The upper laminate 101 is composed of one or more plates, and has at least two hollow structures penetrating the upper laminate 101. In this embodiment, the two hollow structures are preferably two holes 104 with a diameter between 3-10 millimeters that are used for sample injection and the sample or air releasing, respectively. The middle laminate 102 is composed of one or more plates, and has two hollow structures 105 and 106. When the middle laminate 102 is combined with the upper laminate 101 and a lower laminate 103, an imaging chamber 1051 with slot-like shape and biochemical detection area 1061 are formed, respectively. The imaging chamber 1051 can contain and store an added liquid sample or culture medium for cell image capturing and analyzing. The lower laminate 103 is provided with one or more filtering elements 108 and electrode sensing elements 107 that correspond to the location of the biochemical detection area 1061. The filtering element 108 is used to block suspended particles. The electrode sensing element 107 includes an electrode section and electrode terminals. The structure of the electrode section can be electrochemical structure in an X shape, dotted shape, patterns, etc. The electrode terminals can provide instruments or equipment connection in order to perform the measurements and analysis of electrochemistry and impedance.

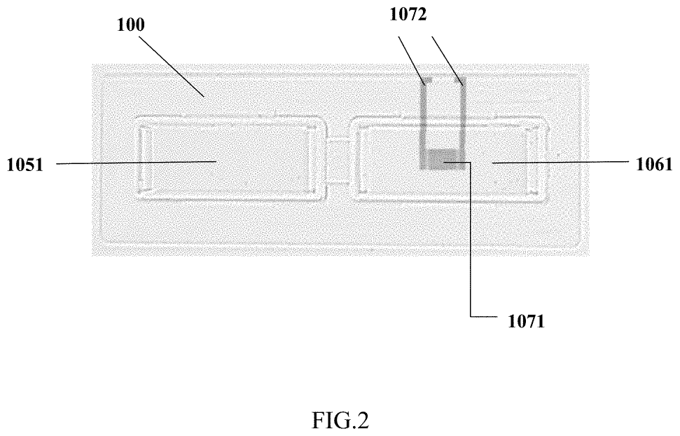

FIG. 2 is a top view photo of the integrated microfluidic chip 100 for cell imaging and biochemical detection. Because the upper laminate 101 is made of a transparent material, the chip 100 can be seen in this photo having two empty slots defined by the hollow structures 105 and 106 to be the imaging chamber 1051 and the biochemical detection area 1061, respectively. In the biochemical detection area 1061, the electrode sensing element 107 disposed on the lower laminate 103 can be seen. The electrode sensing element 107 has the electrode section 1071 and the electrode terminals 1072 to provide instrument or equipment connection so as to perform the measurements and analysis of electrochemistry and impedance.

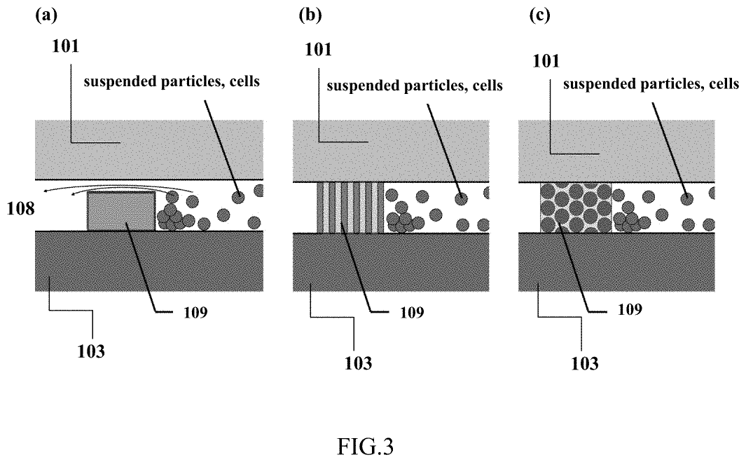

FIG. 3 is the exemplary illustration of the filtering element 108 that the filtering element 108 is a filtering area constructed by one or more microarrays 109 in the form of a slot or pillar (a), fence (b), or sieve (c). The filtering area is mainly used to filter suspended particles (including macromolecules, cells, etc.), which allows the micromolecules in a sample to pass through the filtering element and move into the electrode sensing element 107 where biochemical measurements are performed.

In the integrated chip of the present invention, the imaging chamber 1051 and biochemical detection area 1061 defined by the hollow structures 105 and 106, respectively, can be intercommunicated with each other or be independent. In the design of being intercommunicated with each other, one or more connecting channels are disposed between the imaging chamber 1051 and the biochemical detection area 1061. A filtering element, which is the same as or similar with the aforementioned filtering element, can be disposed in the connecting channels and has a filtering area constructed by one or more microarrays in the form of a pillar, slot, or fence. The image of cells and micromolecules moving into the imaging chamber can be captured by a variety of image capturing devices or microscope imaging systems. The captured image can be further recorded and analyzed after being transmitted to an image analyzing equipment by a receiver.

Electrode sensing element 107, used to perform biochemical and electrical operations or analysis, includes dielectrophoresis control, impedance analysis, and electrochemical measurements, etc. Modifications can be done in the electrode sensing element 107 to assist the detection of biochemical specificity. For example, a molecule for capturing (e.q., the biomolecules such as an antibody, antigen, nucleic acid, protein, etc.) can be modified on the electrode sensing element 107. A target molecule will be bound with the capture molecule when the target to be detected passes through, then the electrochemical measurements for the target molecule can be performed using its electrochemical characteristics. Alternatively, a corresponding detected molecule (e.q., a biomolecule such as an antibody, antigen, nucleic acid, protein, etc., which carries a detectable material) is further introduced and bound with the target molecule captured on the electrode sensing element 107 to perform the electrochemical measurements and analysis for the detected molecule in the electrode section 1071 in accordance with the electrochemical characteristics of the detected molecule.

Embodiment 2: the using method of the integrated microfluidic chip for cell imaging and biochemical detection.

A sample to be tested, which may be a liquid sample or cell culture retrieved from a patient, is injected into the hole 104 defined by the upper laminate 101 of the chip 100. The liquid sample flows into the imaging chamber 1051 and biochemical detection area 1061 that are defined by the hollow structures 105 and 106 of the middle laminate 102, respectively, via microfluidic channels.

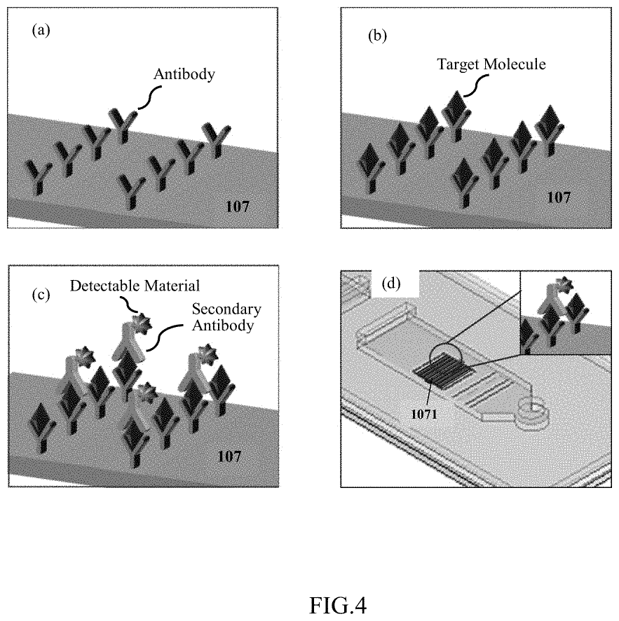

FIG. 4 is an illustration view of an antibody modification and a target molecule detection in the electrode sensing element 107. The embodiment uses an antigen existed in a sample to be tested as the target molecule. As shown in FIG. 4(a), a capture molecule for capturing the specific antigen (e.q., an antibody against the target antigen) is modified first on the electrode sensing element 107. After the fluid sample to be tested is filtered, the target antigen contained in the filtered fluid will be bound with the target molecule on the electrode sensing element 107 (FIG. 4 (b)). A corresponding detected molecule (e.q., a secondary antibody carrying a detectable material) is further introduced and bound with the captured target molecule (FIG. 4 (c)), so as to perform the electrochemical measurements and analysis for the corresponding detected molecule in the electrode section 1071 of the sensing element 107 (FIG. 4(d)) in accordance with the electrochemical characteristics of the detected molecule.

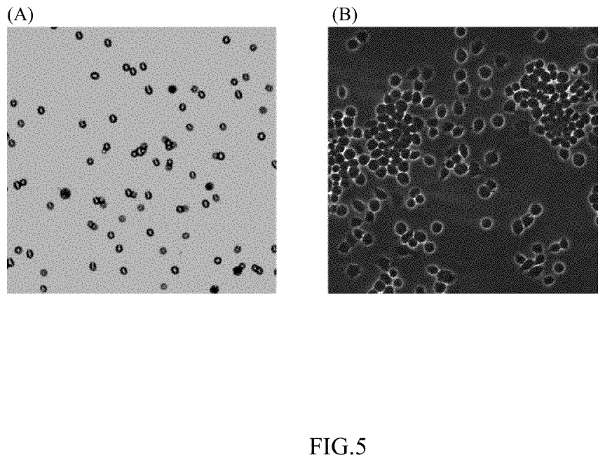

The image of cells in the fluid sample that flows into the imaging chamber 1051 defined by the hollow structure 105 can be captured by a variety of image capturing devices or microscope imaging systems. The image of cells can be analyzed by a microscopic image analysis and interpretation system to determine morphology and pathological conditions of the specific cell. FIG. 5 is a photomicrograph of blood cells (A) and nerve cells (B), which are results that blood and cell culture medium are injected, respectively, into chips. Thereafter, the cells are dispersed in the chamber of the chips, and the chips are then placed in a microscope system for image capturing.

When the fluid sample flowing into the biochemical detection area 1061 passes through the microarray area of the filtering element 108 disposed on the lower laminate 103, the suspended particles (including macromolecules, cells, or debris thereof, etc.) in the fluid sample can be blocked outside the filtering microarrays 109 because of the filtering microarrays 109 in the form of a pillar, slot, fence, or sieve. Accordingly, only the micromolecules in the sample are allowed to pass through the filtering element 108 and move into the electrode sensing element 107 for electrochemical measurements.

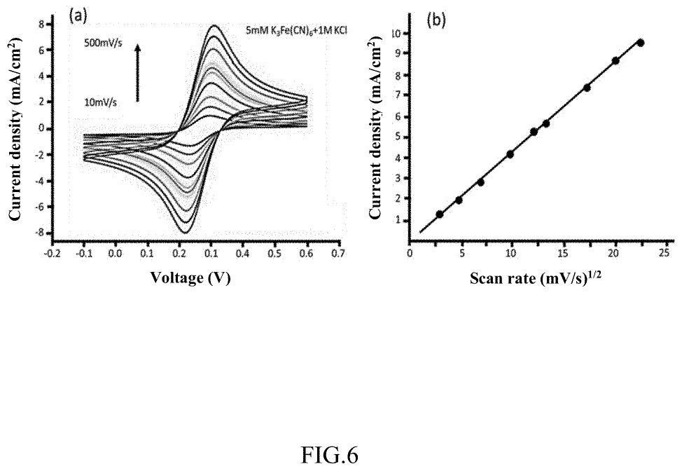

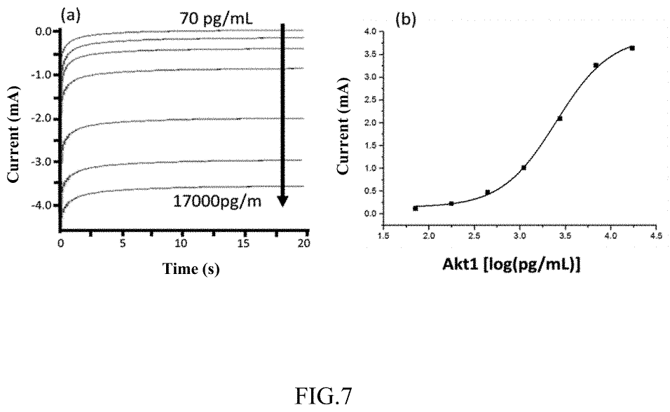

FIG. 6 is a result of the electrochemical measurement of the fluid sample passing through the filtering element 108. The electrochemical measurement is performed in the biochemical detection area 1061 where the solution of potassium hexacyanoferrate (III) is introduced into the chip. The measurement is performed by utilizing the cyclic voltammetry (CV) method in electrochemistry when the electrode terminals 1072 of the chip are connected to an electrochemical sensing instrument. FIG. 7 is a result of the electrochemical measurement of the fluid sample passing through the filtering element 108. The electrochemical measurement is performed in the biochemical detection area 1061 where a different concentration of protein kinase (AKT1) is introduced into the chip, and the measurement is performed by utilizing the chronoamperometry method in electrochemistry when the electrode terminals 1072 of the chip are connected to an electrochemical sensing instrument.

The preferred embodiments above are merely exemplary, the present invention may include various embodiments described in this description and other embodiments. Further, the above-mentioned embodiments are merely illustrations of the present invention and are not limiting. Other equivalent variations and modifications done without departing the spirit disclosed by the present invention shall be included in the claims described below.

While the present invention has been described in terms of what are presently considered to be the most practical and preferred embodiments, it is to be understood that the present invention need not be restricted to the disclosed embodiment. On the contrary, it is intended to cover various modifications and similar arrangements included within the spirit and scope of the appended claims which are to be accorded with the broadest interpretation so as to encompass all such modifications and similar structures. Therefore, the above description and illustration should not be taken as limiting the scope of the present invention which is defined by the appended claims.

What is claimed is:

1. An integrated microfluidic chip for cell imaging and biochemical detection including a sequentially stacked and sealed laminate set, the laminate set comprising:
an upper laminate composed of at least one plate and having at least one hole for sample injection and sample or air discharging;
a middle laminate composed of the at least one plate and including at least two hollow structures penetrating the middle laminate that defining an imaging chamber and a biochemical detection area, respectively; and
a lower laminate composed of the at least one plate and including at least one filtering element and at least one electrode sensing element that disposed on the at least one plate and corresponding to the biochemical detection area,
wherein the at least one filtering element includes a filtering area composed of one or more microarrays, and the at least one electrode sensing element includes an electrode section and an electrode terminal for connecting to devices for inputting current and measuring and analyzing electrochemistry and impedance.

2. The integrated microfluidic chip for cell imaging and biochemical detection of claim 1, wherein the at least one plate of the laminate set is made of a material selected from the groups consisting of light penetrable glass, plastic, and acrylic, and the thickness of each plate is between 50-300 micrometers.

3. The integrated microfluidic chip for cell imaging and biochemical detection of claim 1, further comprising at least one connecting channel,
wherein the at least one connecting channel is disposed between the imaging chamber and the biochemical detection area, and the filtering element includes a filtering area composed of at least one microarray disposed in the connecting channels.

4. The integrated microfluidic chip for cell imaging and biochemical detection of claim 3, wherein the at least one microarray is formed by micro pillars or microbeads arranged in form of pillar, slot or fence.

5. The integrated microfluidic chip for cell imaging and biochemical detection of claim 1, wherein, in the electrode section, a molecule for capturing is modified to be bound with a target molecule.

6. The integrated microfluidic chip for cell imaging and biochemical detection of claim 5, wherein the molecule for capturing is an antibody, antigen, nucleic acid or protein.

7. The integrated microfluidic chip for cell imaging and biochemical detection of claim 1, wherein the imaging chamber is further connected to an image monitoring device that includes a microscopic image receiver and an image analysis device.
